(19) United States
(12) Patent Application Publication
(10) Pub. No.: US 2011/0190622 A1
(43) Pub. Date: Aug. 4, 2011

(54) MAGNETIC RESONANCE IMAGING APPARATUS AND METHOD

(76) Inventor: Mitsuharu MIYOSHI, Tokyo (JP)

(21) Appl. No.: 13/015,365

(22) Filed: Jan. 27, 2011

(30) Foreign Application Priority Data
Jan. 29, 2010 (JP) 2010-019108

(51) Int. Cl. A61B 5/055

(52) U.S. Cl. 600/419

(57) ABSTRACT

A magnetic resonance imaging apparatus executes a pulse sequence for generating a phase shift of each spin, corresponding to a flow rate of the spin to thereby acquire magnetic resonance signals from a subject and determines a position of each blood vessel of the subject, based on each of the magnetic resonance signals. The magnetic resonance imaging apparatus includes a blood vessel position specifying device for specifying a position of each blood vessel, based on a change in signal intensity of the magnetic resonance signal with time and based on a change in the flow rate of the spin with time.

### CROSS REFERENCE TO RELATED APPLICATIONS

This application claims the benefit of Japanese Patent Application No. 2010-019108 filed Jan. 29, 2010, which is hereby incorporated by reference in its entirety.

### BACKGROUND OF THE INVENTION

The present invention relates to a magnetic resonance imaging apparatus for determining a position of each blood vessel of a subject, and a program therefor.

Upon executing a pulse sequence that depends on a blood flow rate, the blood flow rate may be measured in advance. As a method for measuring a blood flow rate, there is known a method for performing a scan for measuring the blood flow rate, causing an operator to find out a blood vessel from each acquired magnetic resonance image and surrounding this blood vessel as an ROI (Region Of Interest). See, for example, Japanese Unexamined Patent Publication 2005-305151.

A problem however arises in that when, for example, the blood vessel is small, the work of setting the region of interest ROI becomes cumbersome. It has thus been desirable to solve the problem.

### BRIEF DESCRIPTION OF THE INVENTION

One aspect of the invention is a magnetic resonance imaging apparatus which executes a pulse sequence for generating a phase shift of each spin, corresponding to a flow rate of the spin to thereby acquire magnetic resonance signals from a subject and determines a position of each blood vessel of the subject, based on each of the magnetic resonance signals, including: a blood vessel position specifying device for specifying a position of each blood vessel, based on a change in signal intensity of the magnetic resonance signal with time and a change in the flow rate of the spin with time.

Another aspect of the invention is a program for a magnetic resonance imaging apparatus which executes a pulse sequence for generating a phase shift of each spin, corresponding to a flow rate of the spin to thereby acquire magnetic resonance signals from a subject and determines a position of each blood vessel of the subject, based on each of the magnetic resonance signals, wherein the program is provided for executing a blood vessel position specifying process for specifying a position of each blood vessel, based on a change in signal intensity of the magnetic resonance signal with time and a change in the flow rate of the spin with time.

The invention is possible to easily decide the position of each blood vessel by a change in signal intensity of each magnetic resonance signal with time and a change in flow rate with time.

Further objects and advantages of the present invention will be apparent from the following description of the preferred embodiments of the invention as illustrated in the accompanying drawings.

### BRIEF DESCRIPTION OF THE DRAWINGS

FIG. 1 is a diagram showing a magnetic resonance imaging apparatus 1 according to a first embodiment of the invention.

FIG. 2 is a diagram illustrating a processing flow of the MRI apparatus 1.

FIGS. 3A and 3B are diagrams depicting a position of a slice SL of a subject 13 and cine images obtained by a phase contrast method.

FIGS. 4A, 4B, and 4C are explanatory diagrams used when determining a maximum value c_max (x, y).

FIGS. 5A and 5B are diagrams showing one example of a binary image representing whether an equation (2) or an equation (3) is established for each position (x, y) of a plane of the slice SL.

FIG. 6 is a diagram schematically illustrating extracted blood vessel regions.

FIG. 7 is a diagram for describing one example of a method for determining whether a correlation in the time direction of data c (x, y, t) is high with respect to pixels adjacent to each other.

FIG. 8 is a diagram showing a flow according to a third embodiment.

FIGS. 9A and 9B are diagrams for explaining the flow according to the third embodiment.

FIG. 10 is a diagram showing a processing flow according to a fourth embodiment.

FIGS. 11A, 11B, and 11C are diagrams for explaining the processing flow according to the fourth embodiment.

FIGS. 12A, 12B, and 12C are diagrams for explaining another processing flow according to the fourth embodiment.

### DETAILED DESCRIPTION OF THE INVENTION

While modes for carrying out the invention will be explained below, the modes for carrying out the invention will not be limited to the following modes or embodiments

### (1) First Embodiment

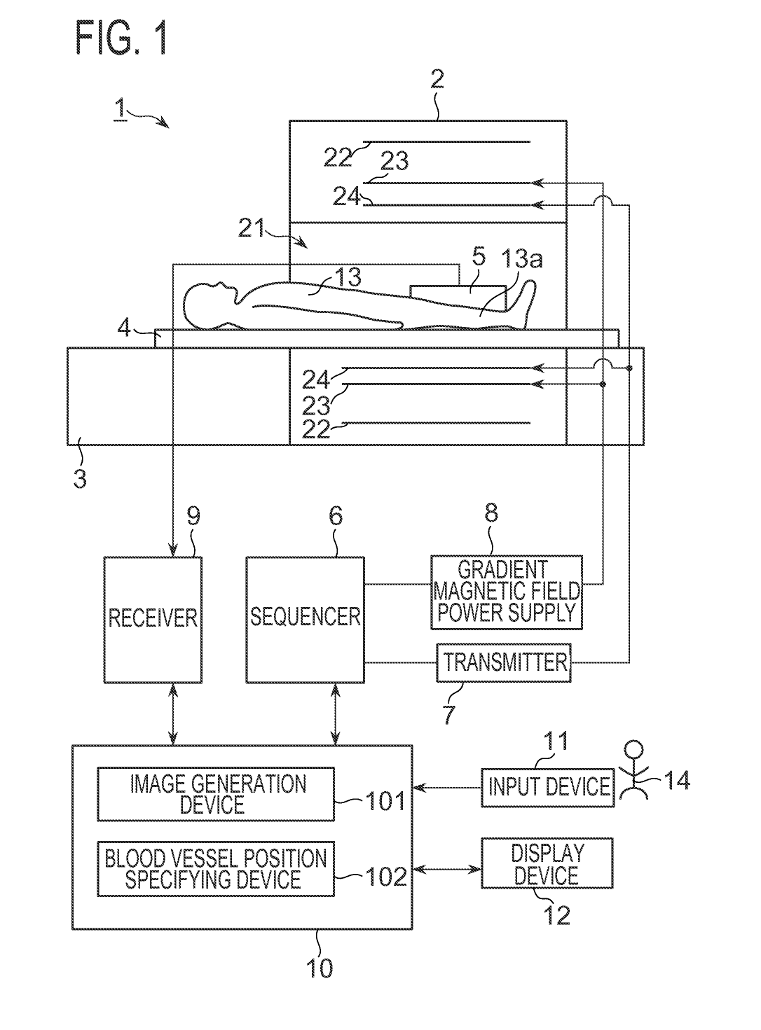

FIG. 1 is a diagram showing a magnetic resonance imaging apparatus 1 according to a first embodiment of the invention.

The magnetic resonance imaging (MRI (Magnetic Resonance Imaging)) apparatus 1 has a magnetic field generator 2, a table 3, a cradle 4, a receiving coil 5, etc.

The magnetic field generator 2 has a bore 21 in which a subject 13 is held, a superconductive coil 22, a gradient coil 23 and a transmitting coil 24. The superconductive coil 22 applies a static magnetic field BO, and the gradient coil 23 applies a gradient magnetic field in a frequency encoding direction, a phase encoding direction and a slice selection direction. The transmitting coil 24 transmits an RF pulse. Incidentally, while the superconductive coil 22 is used in the present embodiment, a permanent magnet may be used instead of the superconductive coil 22.

The cradle 4 is configured so as to be movable from the table 3 to the bore 21. The subject 13 is conveyed to the bore 21 by the cradle 4.

The receiving coil 5 is attached to each leg portion 13a of the subject 13. The receiving coil 5 receives each magnetic resonance signal generated from the subject 13.

The MRI apparatus 1 further has a sequencer 6, a transmitter 7, a gradient magnetic field power supply 8, a receiver 9, a central processing unit 10, an input device 11 and a display device 12.

Under the control of the central processing unit 10, the sequencer 6 transmits information (center frequency, bandwidth and the like) about each RF pulse of a pulse sequence to the transmitter 7 and sends information (intensity of gradient magnetic field, etc.) about a gradient magnetic field to the gradient magnetic field power supply 8.

The transmitter 7 outputs a drive signal for driving the transmitting coil 24, based on the information transmitted from the sequencer 6.

The gradient magnetic field power supply 8 outputs a drive signal for driving the gradient coil 23, based on the information sent from the sequencer 6.

The receiver 9 performs signal processing such as digital conversion on each magnetic resonance signal received by the receiving coil 5 and outputs the same to the central processing unit 10.

The central processing unit 10 controls the operations of respective parts of the MRI apparatus 1 so as to realize various operations of the MRI apparatus 1, such as transmission of necessary information to the sequencer 6 and the display device 12, and reconstruction of an image based on each signal received from the receiver 9. The central processing unit 10 is configured by, for example, a computer. The central processing unit 10 has an image generation device 101 and a blood vessel position specifying device 102. The image generation device 101 generates an image CIₖ (where k=1 to m) to be described later. The blood vessel position specifying device 102 specifies a blood vessel position, based on the image CIₖ (where k=1 to m). Incidentally, the central processing unit 10 functions as the image generation device 101 and the blood vessel position specifying device 102 by executing a predetermined program.

The input device 11 inputs various instructions to the central processing unit 10 in response to the manipulation of an operator 14. The display device 12 displays various information thereon.

The MRI apparatus 1 is configured as described above. A processing flow of the MRI apparatus 1 will next be explained.

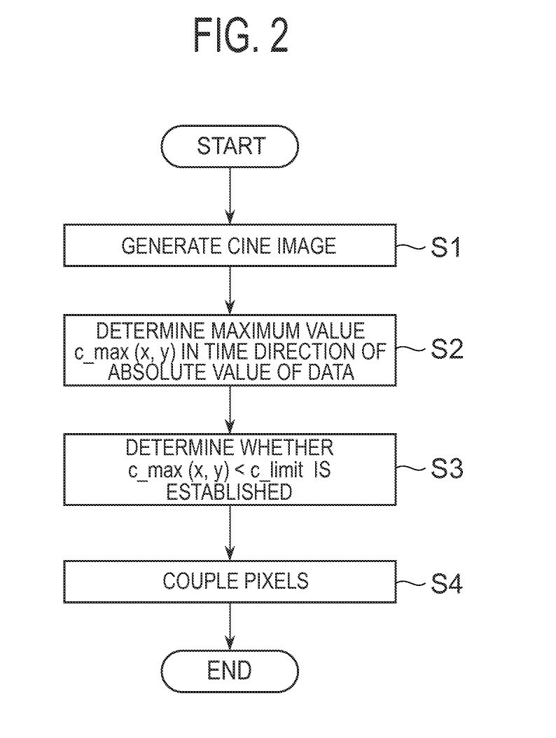

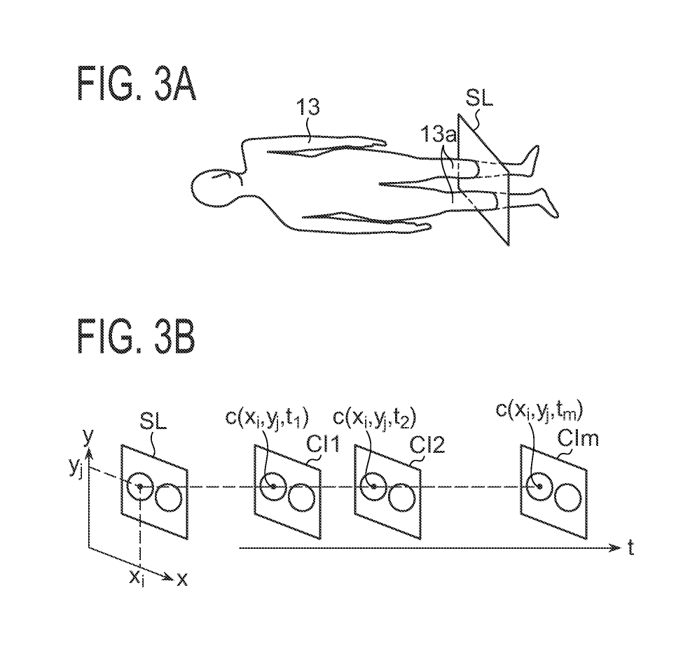

FIG. 2 is a diagram showing the processing flow of the MRI apparatus 1. The processing flow will be explained while referring to FIGS. 3 through 6 as needed upon the description of FIG. 2. Incidentally, the following description will be made of an example in which the position of each of blood vessels in each leg portion 13a of the subject 13 is determined. The invention is however applicable to the case where the positions of blood vessels in an arbitrary portion or region of the subject 13, such as blood vessels in the abdominal region of the subject 13 are determined.

At Step S1, the operator 14 sets a slice SL to the leg portions 13a of the subject 13 (refer to FIG. 3A). Incidentally, although only one sheet of slice SL is set in FIG. 3A, a plurality of sheets of slices may be set.

After the slice SL has been set, a pulse sequence using a phase contrast method is executed to acquire magnetic resonance signals from the slice SL and thereby generate cine images each of which depends on the intensity of the magnetic resonance signal and the flow rate of each spin. In the phase contrast method, the magnitude of a spin's phase shift can be changed according to the flow rate of the spin. Accordingly, information about the flow rate of each spin can be obtained by acquiring the magnetic resonance signals by means of the phase contrast method. In the first embodiment, imaging is performed twice while changing the polarity of a gradient magnetic field at the pulse sequence to thereby acquire complex data f1 and f2. The image generation device 101 (refer to FIG. 1) generates cine images each of which depends on the intensity of each magnetic resonance signal and the flow rate of each spin, based on these complex data f1 and f2 (refer to FIG. 3B).

FIG. 3B is a diagram schematically showing each cine image that depends on the intensity of each magnetic resonance signal and the flow rate of each spin.

An image CIₖ (where k=1 to m) can be obtained by differentiating between the complex data f1 and f2, for example. The position and time of each pixel in the image CIₖ (where k=1 to m) are expressed in (x, y, t). Data represented by each pixel is expressed in c (x, y, t). In the present embodiment, the data c (x, y, t) is defined by the following equation (1):

c(x,y,t)=a(x,y,t)*sin(π*v(x,y,t)/VENC/2) (1)

where a (x, y, t): signal intensity at the position and time (x, y, t) of each pixel, v (x, y, t): flow rate of spin at the position and time (x, y, t) of each pixel, and VENC: gradient amount of velocity encoding.

Data c (xᵢ, yⱼ, t₁), c (xᵢ, yⱼ, t₂), c about pixels at x=xᵢ and y=yⱼ are shown as representative in FIG. 3B. After images CI₁ through CIₘ, have been generated, the operator 14 proceeds to Step S2.

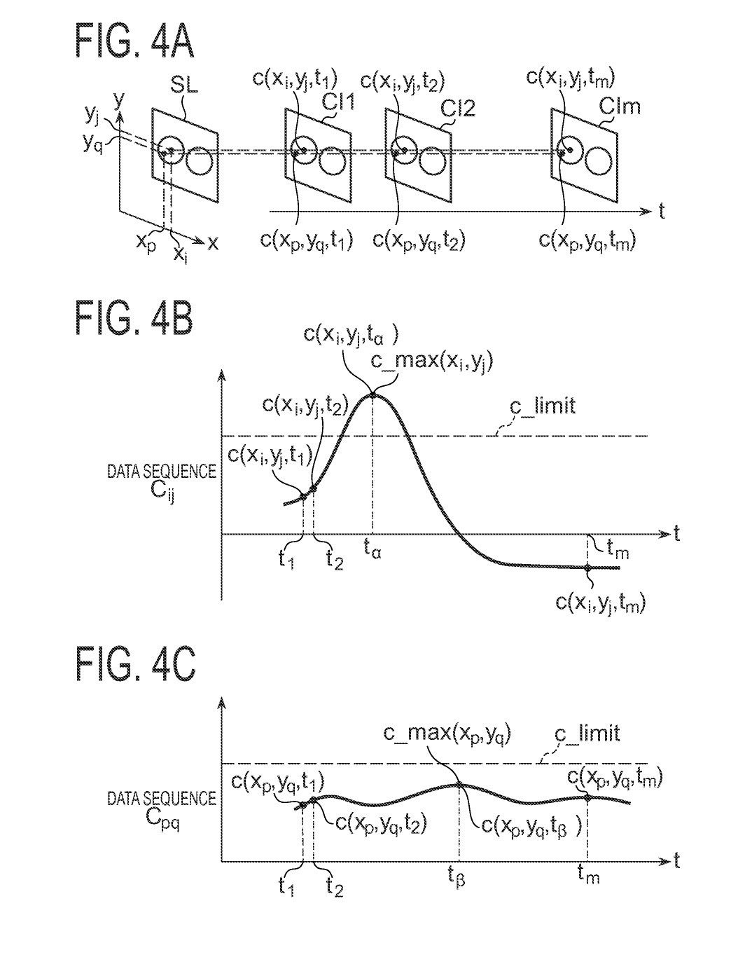

At Step S2, the blood vessel position specifying device 102 (refer to FIG. 1) first calculates the absolute value |c (x, y, t)| of the data c (x, y, t) and determines the maximum value c_max (x, y) in the time direction, of the absolute value |c (x, y, t)| for each position (x, y) of the plane of the slice SL (refer to FIGS. 4A-4C).

FIGS. 4A-4C are explanatory diagrams used when the maximum value c_max (x, y) is determined.

FIG. 4A is a diagram showing the images CI₁ through CIₘ.

When, for example, the maximum value c_max (x, y) at the position (xᵢ, yⱼ) of the plane of the slice SL is determined, the blood vessel position specifying device 102 uses the data c (xᵢ, yⱼ, t) at the position (xᵢ, yⱼ) (refer to FIG. 4B).

FIG. 4B is a diagram showing a data sequence Cᵢⱼ in which the data c (xᵢ, yⱼ, t) at the position (xᵢ, yⱼ) are arranged in time series.

The blood vessel position specifying device 102 determines the absolute value |c (xᵢ, yⱼ, t)| with respect to each data c (xᵢ, yⱼ, t) of the data sequence Cᵢⱼ and determines the maximum value c_max (xᵢ, yⱼ) in the time direction of each absolute value |c (xᵢ, yⱼ, t)|. In FIG. 4B, the absolute value |c (xᵢ, yⱼ, t_{α})| of the data c (xᵢ, yⱼ, t_{α}) at a time t_{α} assumes the maximum value in the time direction. Accordingly, the maximum value c_max (xᵢ, yⱼ) is expressed in the following equation (2):

c_max(xᵢ,yⱼ)=|c(xᵢ,yⱼ,t_{α})| (2)

Thus, the maximum value c_max (xᵢ, yⱼ) in the time direction of the absolute value |c (xᵢ, yⱼ, t)| at the position (xᵢ, yⱼ) can be calculated by the equation (2).

The procedure for calculating the maximum value c_max (xᵢ, yⱼ) at the position (xᵢ, yⱼ) of the plane of the slice SL has been shown in the above description. It is however possible to determine the maximum value c_max (x, y) in the same procedure at any other position (x, y) of the plane of the slice SL. For instance, the maximum value c_max (xₚ, y_{q}) at the position (xₚ, y_{q}) of the plane of the slice SL (refer to FIG. 4A) can be calculated from a data sequence C_{pq} (refer to FIG. 4C) in which data c (xₚ, y_{q}, t) at the position (xₚ, y_{q}) are arranged in time series. The blood vessel position specifying device 102 determines an absolute value|c (xₚ, y_{q}, with respect to the data c (xₚ, y_{q}, t) of the data sequence C_{pq} and determines a maximum value c_max (xₚ, y_{q}) in the time direction of the absolute value |c (xₚ, y_{q}, t)|. In the data sequence C_{pq} of FIG. 4C, an absolute value |c (xₚ, y_{q}, t_{β})| of data c (xₚ, y_{q}, t_{β}) at a time t_{β} assumes a maximum value in the time direction. Accordingly, the maximum value c_max (xₚ, y_{q}) is expressed in the following equation (3):

c_max(xₚ,y_{q})=|c(xₚ,y_{q},t_{β})| (3)

The maximum value c_max (xₚ, y_{q}) in the time direction of the absolute value |c (xₚ, y_{q}, t)| at the position (xₚ, y_{q}) can thus be calculated by the equation (3).

After the maximum value c_max (x, y) in the time direction of the absolute value |c (x, y, t)| of the data c (x, y, t) has been determined for each position (x, y) of the plane of the slice SL according to the above procedure, the operator 14 proceeds to Step S3.

At Step S3, the blood vessel position specifying device 102 determines whether the maximum value c_max (x, y) determined at Step S2 is smaller than a threshold value c_limit. Generally, the maximum value c_max (x, y) tends to become large in the case of each magnetic resonance signal arising from the blood vessel, whereas the maximum value c_max (x, y) tends to become small in the case of each magnetic resonance signal arising from a stationary tissue and signals (noise) from outside the body of the subject 13. It can thus be determined that when the following equation (4) is established, it represents the magnetic resonance signal arising from the stationary tissue or the noise. On the other hand, it can be determined that when the following equation (5) is established, there is a high possibility that it represents the magnetic resonance signal arising from the blood vessel

c_max(x,y)<c_limit (4)

c_max(x,y)≦c_limit (5)

Incidentally, c_limit can be optimized by iterative calculation or the like.

At the position (xᵢ, yⱼ) of the plane of the slice SL, for example, the maximum value c_max (xᵢ, yⱼ) is larger than the threshold value c_limit as shown in FIG. 4B. Thus, since the equation (5) is established at the position (xᵢ, yⱼ) of the plane of the slice SL, it is considered that the possibility of the blood vessel is high.

On the other hand, the maximum value c_max (xₚ, y_{q}) is smaller than the threshold value c_limit as the position (xₚ, y_{q}) of the plane of the slice SL as shown in FIG. 4C. Thus, since the equation (4) is established at the position (xₚ, y_{q}) of the plane of the slice SL, it is considered that the possibility of the stationary tissue or noise is high (i.e., the possibility of the blood vessel is low).

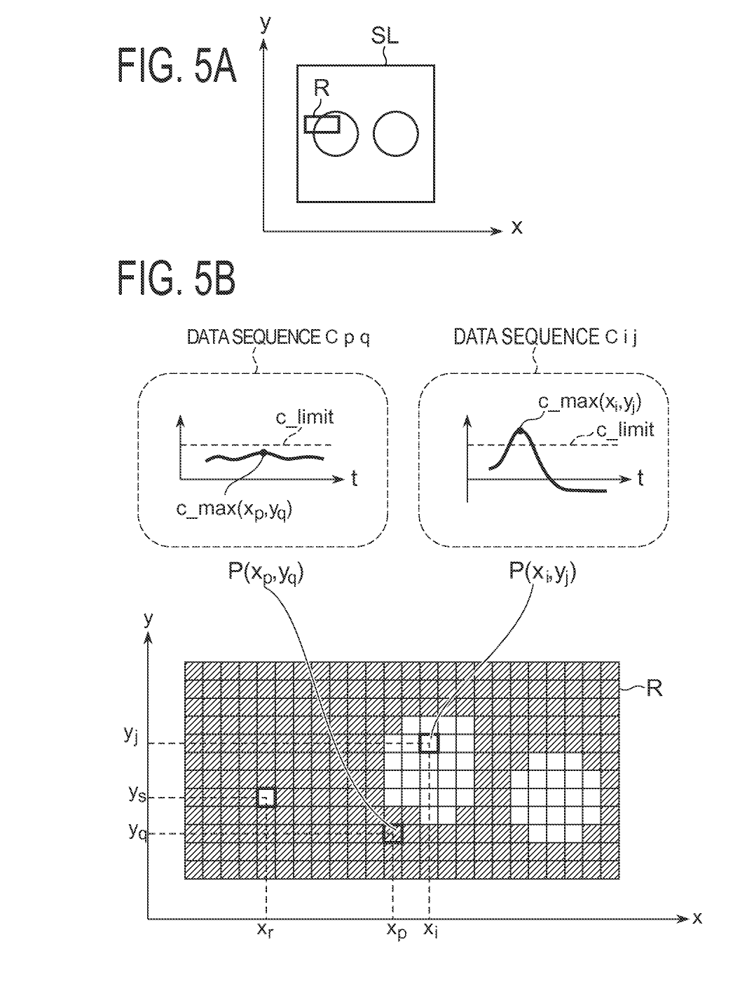

Similarly, it is determined whether the equation (4) or (5) is established with respect to any other position (x, y) of the plane of the slice SL (refer to FIGS. 5A and 5B).

FIGS. 5A and 5B are diagrams showing one example of a binary image representing whether the equation (4) or (5) is established for each position (x, y) of the plane of the slice SL.

FIG. 5A is a diagram showing the plane of the slice SL, and FIG. 5B shows the binary image representing whether the equation (4) or (5) is established at each position lying within a partial region R of the slice SL shown in FIG. 5A.

In FIG. 5B, each pixel shown diagonally shaped indicates the position where the equation (4) is established in the region R of the slice SL (i.e., position where the possibility of the stationary tissue or that of the outside of the body of the subject is high). Since the maximum value c_max (xₚ, y_{q}) calculated based on the data sequence C_{pq} is established in the equation (4) at the position (xₚ, y_{q}), for example, a pixel P (xₚ, y_{q}) indicates the position where the possibility of the stationary tissue or the outside of the body of the subject is high.

On the other hand, each open pixel indicates the position (x, y) (i.e., position at which the possibility of the blood vessel is high) where the equation (5) is established in the region R of the slice SL. Since the maximum value c_max (xᵢ, yⱼ) calculated based on the data Cᵢⱼ satisfies the equation (5) at the position (xᵢ, yⱼ), for example, a pixel P (xᵢ, yⱼ) indicates the position where the possibility of the blood vessel is high.

Thus, each pixel (open pixel) high in the possibility of the blood vessel can be specified by determining whether the equation (4) is established. While each pixel (open pixel) high in the possibility of the blood vessel is shown in the partial region R of the slice SL in FIGS. 5A and 5B for convenience of explanation, pixels high in the possibility of blood vessels are actually specified over the whole region of the slice SL. After Step S3 has been executed, the operator 14 proceeds to Step S4.

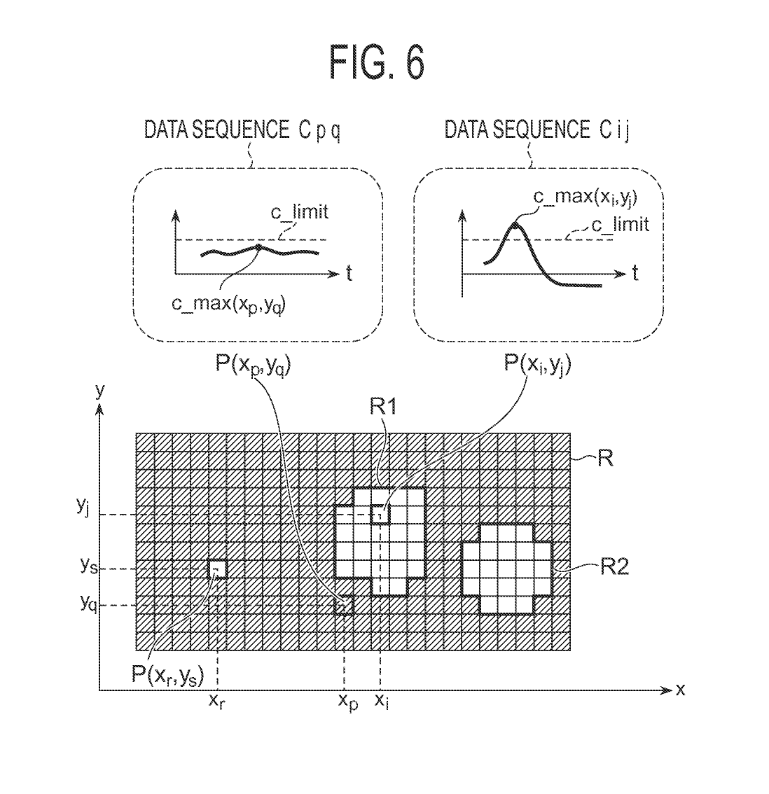

At Step S4, the blood vessel position specifying device 102 (refer to FIG. 1) couples pixels adjacent to each other from within the pixels (open pixels shown in FIGS. 5A and 5B) high in the possibility of the blood vessels and extracts blood vessel regions (refer to FIG. 6).

FIG. 6 is a diagram schematically showing the extracted blood vessel regions.

Coupling the adjoining pixels to each other enables the extraction of the blood vessel regions R1 and R2.

Incidentally, since the pixel P (xᵣ, yₛ) is of an open pixel in FIG. 6, it corresponds to a pixel judged to be high in the possibility of the blood vessel at Step S3. The pixel P (xᵣ, yₛ) is however surrounded by pixels of a stationary tissue or pixels lying outside the body of the subject 13 (pixels shown diagonally shaded in FIG. 6). Thus, even in the case of the pixels judged to be high in the possibility of the blood vessels, it is considered that the possibility of the blood vessel is low where the pixel is surrounded by the pixels of the stationary tissue or the pixels outside the body of the subject 13. Therefore, the pixels are judged not to correspond to the blood vessels.

The flow is ended in the above-described manner.

Generally, the maximum value c_max (x, y) tends to become large in the case of each magnetic resonance signal arising from the blood vessel, whereas the maximum value c_max (x, y) tends to become small in the case of the signal (noise) lying outside the body of the subject 13. Accordingly, the blood vessel regions can be extracted by calculating the maximum values c_max (x, y) every position (x, y) of the slice SL from the image CIₖ that depends on the signal intensity and the flow rate.

In the first embodiment, the position of each blood vessel is specified based on the maximum value c_max (x, y) in the time direction of the absolute value |c (x, y, t)| of the data c (x, y, t). Since there is however no need to determine the absolute value |c (x, y, t)| where the data c (x, y, t) is not brought to a negative value, the position of the blood vessel may be specified based on the maximum value in the time direction of the data c (x, y, t). Further, the data c (x, y, t) is weighted and the position of each blood vessel may be specified based on the weighted data c (x, y, t).

Incidentally, the data c (x, y, t) is of data that depends on the signal intensity a (x, y, t) and the flow rate v (x, y, t) (refer to the equation (1)). Accordingly, the position of each blood vessel may be specified by determining the signal intensity a (x, y, t) and the flow rate v (x, y, t) without determining the data c (x, y, t) and by analyzing a change in the signal intensity a (x, y, t) with time and a change in the flow rate v (x, y, t) with time.

### (2) Second Embodiment

A second embodiment will be explained while referring to the flow shown in FIG. 2. Incidentally, since the second embodiment is identical to the first embodiment in terms of Steps S1 through S3, the description of Steps S1 through S3 is omitted and only Step S4 will therefore be explained.

At Step S4 of the second embodiment, the blood vessel position specifying device 102 (refer to FIG. 1) determines whether a correlation in the time direction of data c (x, y, t) is high with respect to pixels adjacent to each other out of the pixels (open pixels shown in FIGS. 5A and 5B) judged to be high in the possibility of each blood vessel at Step S3.

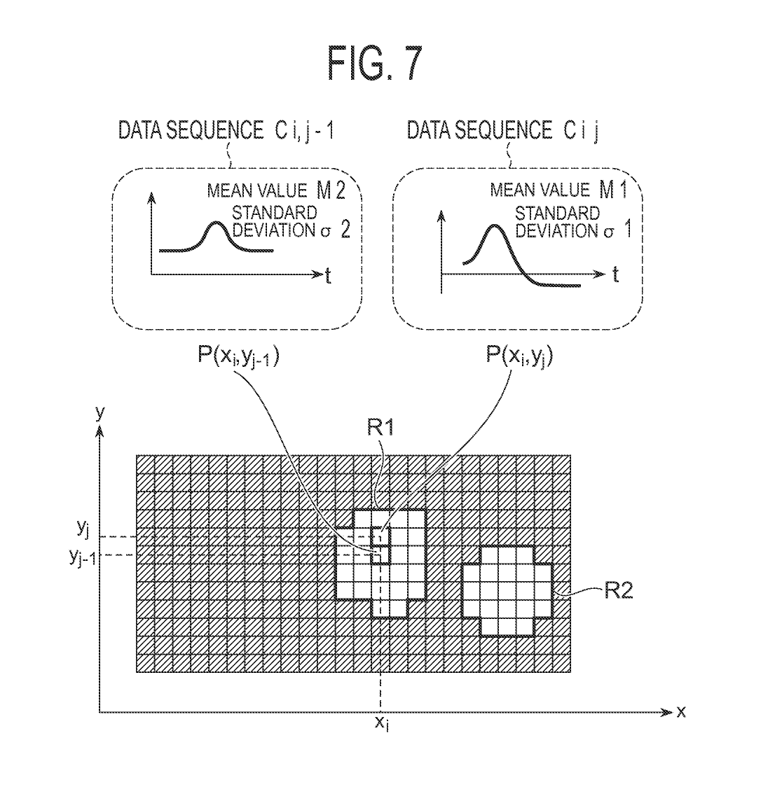

FIG. 7 is a diagram for explaining one example of a method for determining whether a correlation in the time direction of data c (x, y, t) is high with respect to pixels adjacent to each other.

When the correlation in the time direction of the data c (x, y, t) is determined with respect to, for example, adjoining pixels P (xᵢ, yⱼ) and P (xᵢ, yⱼ₋₁), a coefficient of correlation COR between a data sequence Cᵢⱼ at the pixel P (xᵢ, yⱼ) and a data sequence C_{i, j-1} at the pixel P (xᵢ, yⱼ₋₁) may be calculated. Generally, the coefficient of correlation COR in the time direction of the data c (x, y, t) tends to become large at the pixel of each blood vessel. Therefore, when the correlation coefficient COR is large (e.g., COR>0.8), the corresponding pixel is considered to be a blood vessel pixel. On the other hand, when the correlation coefficient COR is small (e.g., COR≦0.8), it is considered that the possibility of the blood vessel pixel is low. Thus, even if it is erroneously determined at Step S3 that the pixel non-corresponding to the blood vessel is of a pixel high in the possibility of the blood vessel, it can be eliminated out of the pixels of the blood vessels by calculating the correlation coefficient COR at Step S4. In the second embodiment, when it is determined that the correlation in the time direction of the data c (x, y, t) is high, the adjoining pixels are coupled to one another and thereby each blood vessel region is extracted. It is thus possible to extract the blood vessel regions with a high degree of accuracy.

Incidentally, since the vein is slower than the artery in flow rate, the value of the correlation coefficient COR becomes small depending on the flow rate of the vein regardless of the presence of each pixel for the vein, so that it may be judged not to be a blood vessel pixel. Although the pixel P (xᵢ, yⱼ) has been determined to correspond to the blood vessel pixels where, for example, the region R1 of the blood vessel is of a vein region, the pixel P (xᵢ, yⱼ₋₁) may be judged not to be the blood vessel pixels. As a method for avoiding such misjudgments, there is considered, for example, a case where a mean value M1 of the data sequence Cᵢⱼ at the pixel P (xᵢ, yⱼ) and a standard deviation 61 thereof, and a mean value M2 of the data sequence at the pixel P (xᵢ, yⱼ₋₁) and a standard deviation σ2 thereof are determined and thereby an F test and a T test are performed. It has generally been known that when the vein pixels are compared with each other, the mean values of data sequences of data c (x, y, t) tend to approximately the same value, and the mean values of data sequences of data c (x, y, t) also tend to approximately the same value. Therefore, when the F test and T test are allowed to pass, the corresponding pixel can be judged to be the vein pixel. Thus, when the F test and the T test are allowed to pass, even though the pixel P (xᵢ, yⱼ₋₁) is eliminated from the blood vessel pixels, where the determination from the value of the correlation coefficient COR is made, the pixel P (xᵢ, yⱼ₋₁) can also be determined to correspond to the vein pixel, thereby making it possible to extract blood vessel regions with a higher degree of accuracy.

### (3) Third Embodiment

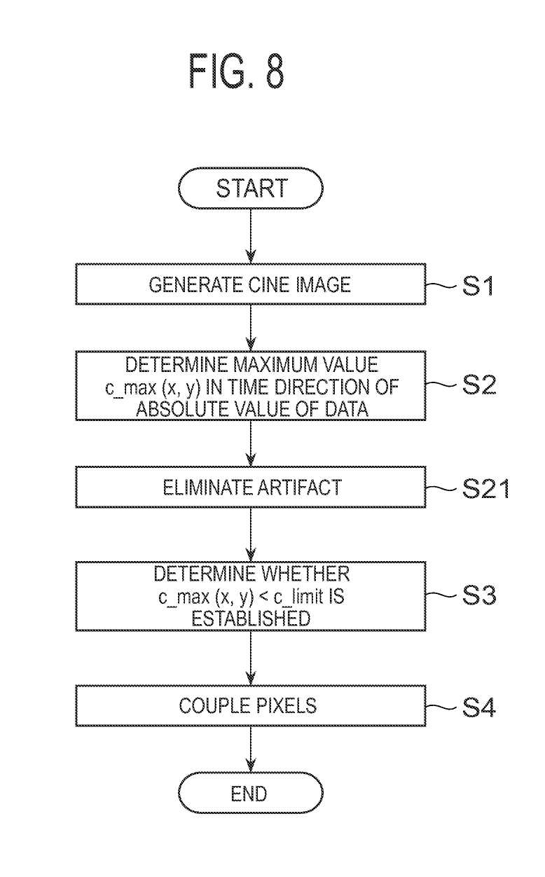

A third embodiment will be explained referring to a flow shown in FIG. 8.

FIG. 8 is a diagram showing the flow according to the third embodiment.

Since the third embodiment is identical to the first embodiment in terms of Steps S1 and S2, the description of Steps S1 and S2 will be omitted. After Step S2 has been ended, the operator 14 proceeds to Step S21.

At Step S21, the blood vessel position specifying device 102 (refer to FIG. 1) eliminates pixels high in the possibility of artifacts. Incidentally, although the following description will be made of an example in which a pixel P (xᵢ, yⱼ) is high in the possibility of artifacts, it is possible to determine by a similar method whether other pixels are also artifacts.

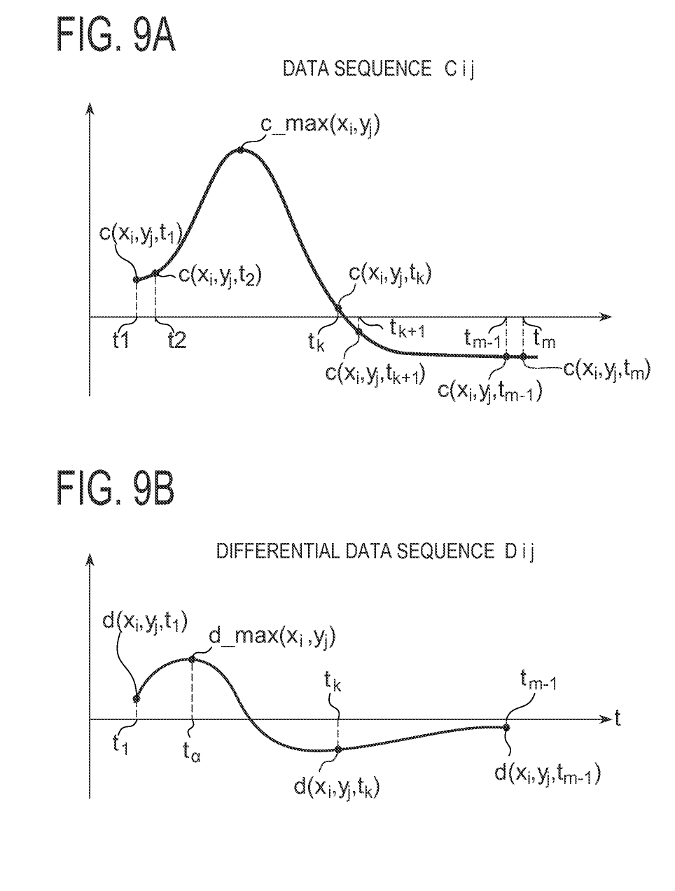

A differentiation between data c (xᵢ, yⱼ, t) in the time direction is first performed on a data sequence Cᵢⱼ (refer to, for example, FIG. 4(b)) at the pixel P (xᵢ, yⱼ) (refer to FIGS. 9A and 9B).

FIG. 9A is a diagram schematically showing the data sequence Cᵢⱼ at the pixel P (xᵢ, yⱼ), and FIG. 9B is a diagram schematically showing a differential data sequence Dᵢⱼ obtained by differentiating between the data c (xᵢ, yⱼ, t) of the data sequence Cᵢⱼ in the time direction.

Data (hereinafter called “differential data”) d (xᵢ, yⱼ, tₙ) at a time tₙ (where n=1 to m−1) of the differential data sequence Dᵢⱼ is expressed in the following equation (6) using data c (xᵢ, yⱼ, tₙ₊₁) and c (xᵢ, yⱼ, tₙ):

d(xᵢ,yⱼ,tₙ)=c(xᵢ,yⱼ,tₙ₊₁)−c(xᵢ,yⱼ,tₙ) (6)

Thus, for example, differential data d (xᵢ, yⱼ, tₖ) at a time tₖ is expressed in the following equation (6′) through the equation (6):

d(xᵢ,yⱼ,tₖ)=c(xᵢ,yⱼ,tₖ₊₁)−c(xᵢ,yⱼ,tₖ) (6′)

After the differential data d (xᵢ, yⱼ, tₙ) has been determined, the maximum value d_max (xᵢ, yⱼ) in the time direction, of the absolute value |d (xᵢ, yⱼ, tₙ)| of the differential data d (xᵢ, yⱼ, tₙ) is determined. In FIG. 9B, the absolute value |d(xᵢ, yⱼ, t_{α})| of differential data at a time t_{α} assumes the maximum value in the time direction. Accordingly, the maximum value d_max (xᵢ, yⱼ) is expressed in the following equation (7):

d_max(xᵢ,yⱼ)=|d(xᵢ,yⱼ,t_{α}) (7)

Next, the maximum value d_max (xᵢ, yⱼ) at the differential data sequence Dᵢⱼ and the maximum value c_max (xᵢ, yⱼ) at the data sequence Cᵢⱼ are compared with each other. In general, the data c (x, y, t) changes smoothly and gently in the time direction in the case of a blood flow. Thus, in the case of blood vessel pixels, the maximum value d_max (xᵢ, yⱼ) at the differential data sequence Dᵢⱼ necessarily results in a value smaller than the maximum value c_max (xᵢ, yⱼ) at the data sequence Cᵢⱼ. On the other hand, the maximum value d_max (xᵢ, yⱼ) is not necessarily brought to the small value in the case of abnormal signals such as artifacts. Thus, when the following equation (8) is established, it can be judged to be indicative of a magnetic resonance signal arising from an artifact. On the other hand, when an equation (9) is established, it can be determined that the possibility of a magnetic resonance signal arising from the blood vessel is high.

d_max(xᵢ,yⱼ)>const1*c_max(xᵢ,yⱼ) (8)

d_max(xᵢ,yⱼ)≦const1*c_max(xᵢ,yⱼ) (9)

Incidentally, const1 is of an experience value.

Thus, the artifacts can be eliminated by determining whether the equation (8) is established, thereby making it possible to extract the blood vessels with a higher degree of accuracy. After the elimination of the artifacts, the operator 14 proceeds to Steps S3 and S4, where a blood vessel region is extracted.

Incidentally, in the above description, the artifacts are eliminated based on the result of comparison between the maximum value d_max (xᵢ, yⱼ) at the differential data sequence Dᵢⱼ and the maximum value c_max (xᵢ, yⱼ) at the data sequence Cᵢⱼ. However, a standard deviation d_std (xᵢ, yⱼ) of the differential data sequence Dᵢⱼ and a standard deviation d_std (xᵢ, yⱼ) of the data sequence Cᵢⱼ are determined and thereby the artifacts may be eliminated based on the result of comparison between these standard deviations d_std (xᵢ, yⱼ) and c_std (xᵢ, yⱼ). Since the data c (xᵢ, yⱼ, t) changes smoothly and gently in the time direction in the case of the blood flow, the standard deviation d_std (xᵢ, yⱼ) of the differential data sequence Dᵢⱼ necessarily results in a value smaller than the standard deviation c_std (xᵢ, yⱼ) of the data sequence Cᵢⱼ. On the other hand, the standard deviation d_std (xᵢ, yⱼ) is not necessarily brought to a small value in the case of an abnormal signal such as an artifact. Thus, when the following equation (10) is established, it can be judged to be indicative of a magnetic resonance signal arising from the artifact. On the other hand, when an equation (11) is established, it can be judged that the possibility of a magnetic resonance signal arising from the blood vessel is high.

d_std(xᵢ,yⱼ)>const2*c_std(xᵢ,yⱼ) (10)

d_std(xᵢ,yⱼ)≦const2*c_std(xᵢ,yⱼ) (11)

Incidentally, const2 indicates an experience value.

Thus, the artifacts can be eliminated even by comparing the standard deviations, thereby making it possible to perform the extraction of the blood vessels with a higher degree of accuracy.

The artifacts may be eliminated in consideration of both the result of comparison between the maximum values d_max (xᵢ, yⱼ) and c_max (xᵢ, yⱼ) and the result of comparison between the standard deviations d_std (xᵢ, yⱼ) and c_std (xᵢ, yⱼ).

### (4) Fourth Embodiment

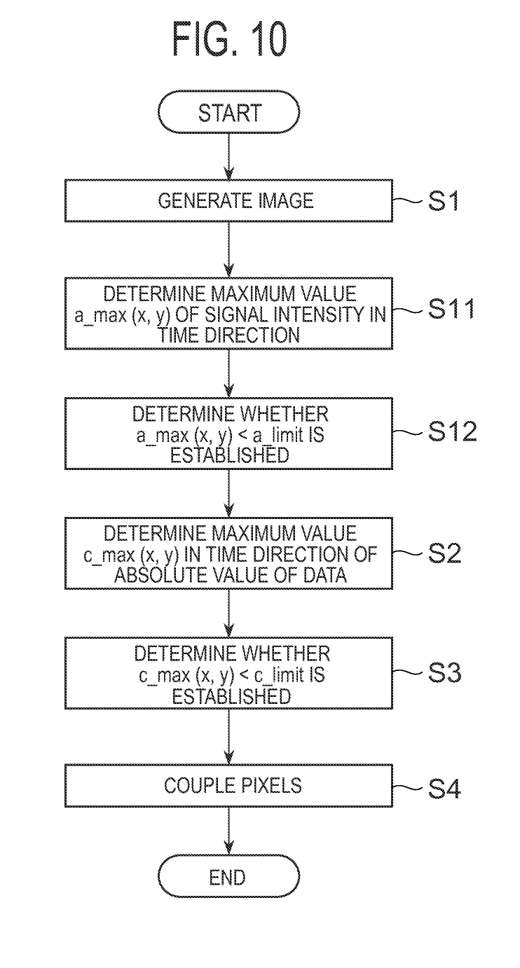

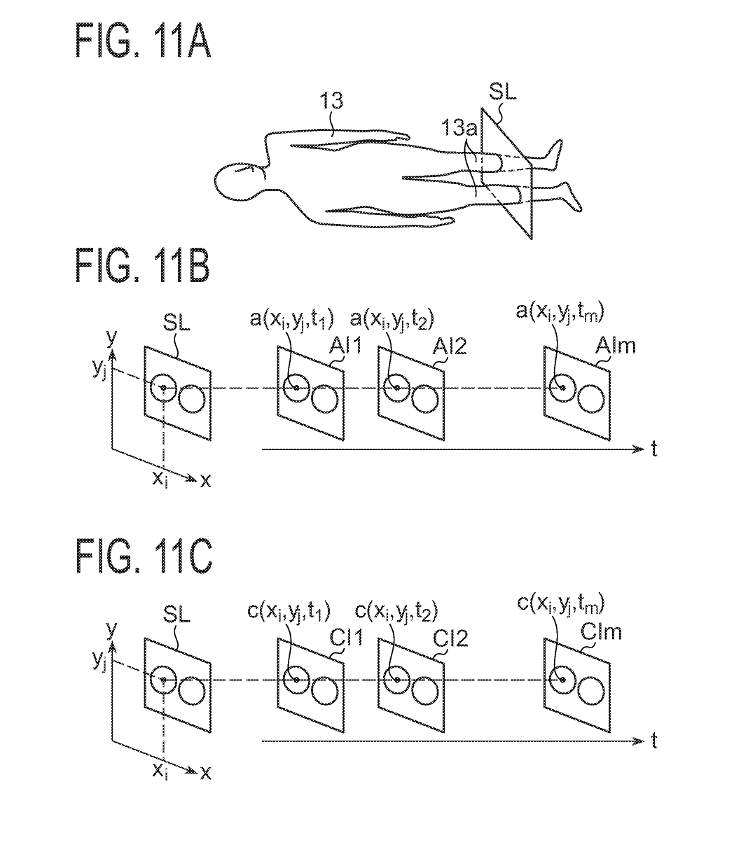

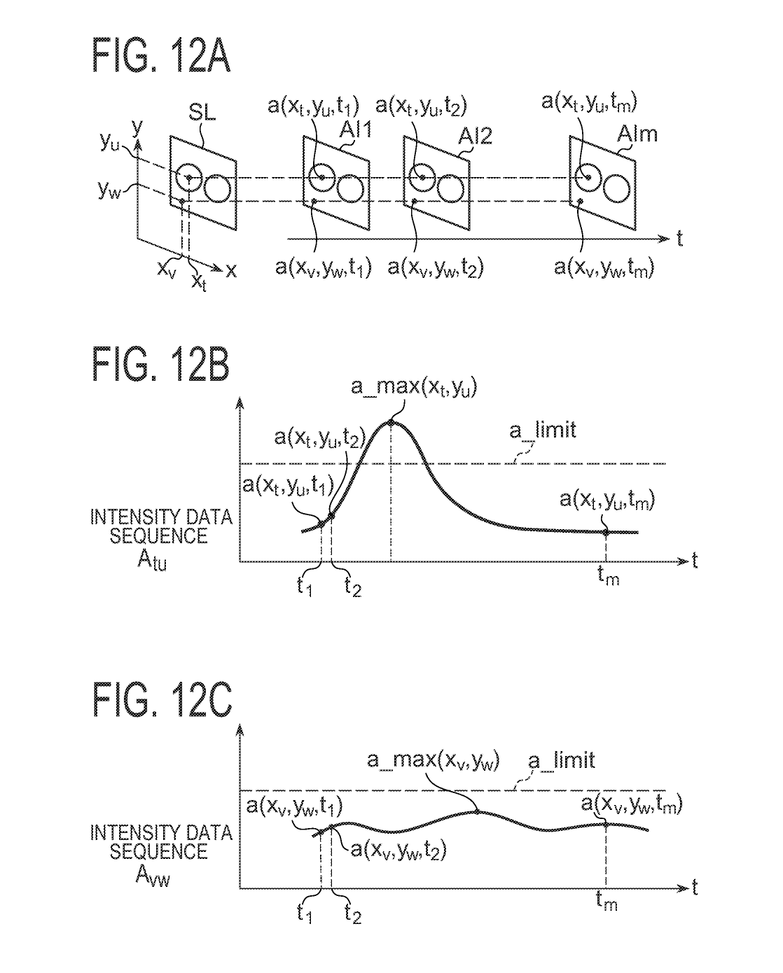

FIG. 10 is a diagram showing a processing flow according to a fourth embodiment. Incidentally, the processing flow of FIG. 10 will be explained while referring to FIGS. 11 and 12 as needed upon its description.

At Step S1, a slice SL is first set (refer to FIG. 11A). After the slice SL has been set, a pulse sequence using a phase contrast method is executed to acquire magnetic resonance signals from the slice SL and thereby generate cine images each indicative of the intensity of the magnetic resonance signal, and cine images each of which depends on the intensity of the magnetic resonance signal and the flow rate of each spin. In the phase contrast method, the magnitude of a spin's phase shift can be changed according to the flow rate of the spin. Accordingly, information about the flow rate of each spin can be obtained by acquiring the magnetic resonance signals by means of the phase contrast method. In the fourth embodiment, imaging is performed twice while changing the polarity of a gradient magnetic field at the pulse sequence to thereby acquire complex data f1 and f2. The image generation device 101 (refer to FIG. 1) generates cine images each of which depends on the intensity of the magnetic resonance signal and the flow rate of the spin, based on these complex data f1 and f2. FIG. 11B shows a intensity image AIₖ (where k=1 to m) indicative of a signal intensity, and FIG. 11C shows an image CIₖ (where k=1 to m) that depends on the signal intensity and the flow rate. The intensity image AIₖ (where k=1 to m) can be obtained as, for example, an absolute value |f1|(=|f2|) of complex data. The image CIₖ (where k=1 to m) can be obtained by, for example, differentiating the complex data f1 and f2.

Positions and times of respective pixels of the intensity images AI₁ through AIₘ are expressed in (x, y, t). A signal intensity represented by each pixel is expressed in a (x, y, t). Signal intensities a (xᵢ, yⱼ, t₁), a (xᵢ, yⱼ, t₂), . . . a (xᵢ, yⱼ, tₘ) of pixels at x=xᵢ and y=yⱼ are typically shown in FIG. 11B. Incidentally, since the images CI₁ through CIₘ each of which depends on the signal intensity and the flow rate are similar to those employed in the first embodiment, the description thereof will be omitted.

After the intensity images AI₁ through AIₘ, and the images CI₁ through CIₘ each of which depends on the signal intensity and the flow rate, have been generated, the operator 14 proceeds to Step S11.

At Step S11, the maximum value a_max (x, y) in the time direction, of the signal intensity a (x, y, t) is calculated for each position (x, y) of a plane of the slice SL using the intensity images AI₁ through AIₘ.

FIGS. 12A-12C are explanatory diagrams used when the maximum value a_max (x, y) of the signal intensity a (x, y, t) in the time direction is calculated.

FIG. 12A is a diagram showing cine images for intensity images AI₁ through AIₘ.

When, for example, the maximum value a_max (xₜ, yᵤ) in the time direction of a signal intensity a (xₜ, yᵤ, t) at a position (xₜ, yᵤ) of the plane of the slice SL is calculated, signal intensities a (xₜ, yᵤ, t₁) through a (xₜ, yᵤ, tₘ) at the position (xₜ, yᵤ) of the plane of the slice SL may be taken out from the intensity images AI₁ through AIₘ (refer to FIG. 12B).

FIG. 12B shows an intensity data sequence Aₜᵤ indicative of changes in the signal intensities a (xₜ, yᵤ, t₁) through a (xₜ, yᵤ, tₘ) with time. Determining the intensity data sequence Aₜᵤ enables the calculation of the maximum value a_max (xₜ, yᵤ) in the time direction of the signal intensity at the position (xₜ, yᵤ) of the plane of the slice SL.

The procedure of calculating the maximum value a_max (xₜ, yᵤ) in the time direction of the signal intensity a (xₜ, yᵤ, t) at the position (xₜ, yᵤ) of the plane of the slice SL has been shown in the above description. The maximum value in the time direction of the signal intensity a (x, y, t) at each of other positions (x, y) of the plane of the slice SL can also, however, be determined in a similar procedure. The maximum value a_max (x_{y}, y_{w}) in the time direction of the signal intensity a (xᵥ, y_{w}, t) at a position (xᵥ, y_{w}) (refer to FIG. 12A) of the plane of the slice SL is shown in, for example, FIG. 12C. The maximum value a_max (xᵥ, y_{w}) can be determined from a intensity data sequence A_{vw} of the signal intensities a (xᵥ, y_{w}, t₁) through a (xᵥ, y_{w}, tₘ) at the position (xᵥ, y_{w}) of the plane of the slice SL.

After the maximum value a_max (x, y) of the signal intensity a (x, y, t) in the time direction has been determined in the above procedure for each position (x, y) of the plane of the slice SL, the operator 14 proceeds to Step S12.

At Step S12, it is determined whether the maximum value a_max (x, y) of the signal intensity in the time direction, which has been determined at Step S11, is smaller than a threshold value a_limit. In general, the maximum value a_max (x, y) of the signal intensity in the time direction tends to become large within the body of the subject 13, whereas the maximum value a_max (x, y) of the signal intensity in the time direction tends to become small outside the body of the subject 13. It can thus be determined that when the following equation (12) is established, it is possible to judge that the possibility of noise is high. On the other hand, it can be determined that when an equation (13) is established, it is possible to judge that the possibility of a signal (magnetic resonance signal arising from within the body of the subject 13) other than noise is high.

a_max(x,y)<a_limit (12)

a_max(x,y)≦a_limit (13)

Incidentally, a_limit can be optimized by iterative calculation or the like.

At the position (xₜ, yᵤ) of the plane of the slice SL, for example, the maximum value a_max (xₜ, yᵤ) in the time direction of the signal intensity is larger than the threshold value a_limit as shown in FIG. 12B. Thus, since the equation (13) is established at the position (xₜ, yᵤ) of the plane of the slice SL, it is considered that the possibility of the signal (magnetic resonance signal arising from within the body of the subject 13) other than noise is high.

On the other hand, the maximum value a_max (xᵥ, y_{w}) in the time direction of the signal intensity is smaller than the threshold value a_limit at the position (xᵥ, y_{w}) of the plane of the slice SL as shown in FIG. 12C. Thus, since the equation (12) is established at the position (xᵥ, y_{w}) of the plane of the slice SL, it is considered that the possibility of noise (magnetic resonance signal outside the body of the subject) is high.

Similarly, it is determined whether the equation (12) or (13) is established with respect to any other position (x, y) of the plane of the slice SL. Thus, noise can efficiently be eliminated by determining whether the maximum value a_max (x, y) of the signal intensity in the time direction is greater than or equal to the threshold value a_limit over the whole plane of the slice SL. After the end of Step S12, the operator 14 proceeds to Step S2.

Since Steps S2 through S4 are similar to those employed in the first embodiment, the description thereof will be omitted.

In the fourth embodiment, it has been determined at Step S12 whether the maximum value a_max (x, y) of the signal intensity in the time direction is greater than or equal to the threshold value a_limit over the whole plane of the slice SL. Accordingly, noise can efficiently be eliminated before the pixels are coupled to each other at Step S4, thereby making it possible to extract blood vessel regions with a higher degree of accuracy.

The fourth embodiment has explained the example in which the intensity image AIₖ is generated in addition to the image CIₖ that depends on the signal intensity and the flow rate. A flow rate image indicative of the flow rate of each spin may however be generated in addition to the intensity image AIₖ (or instead of the intensity image AIₖ). Since the vein is slower in flow rate than the artery, it is possible to recognize by generation of the flow rate image whether the extracted blood vessel region is of the venous blood vessel or the arterial blood vessel.

Incidentally, the data c (x, y, t) expressed in the equation (1) has been used in each of the first through fourth embodiments. Since, however, the data depends on the flow rate v (x, y, t) and the signal intensity a (x, y, t), data different from the data c (x, y, t) may be used. For example, data p (x, y, t) obtained by multiplying the signal intensity a (x, y, t) and the flow rate v (x, y, t) by each other may be used. In this case, the data p (x, y, t) is expressed in the following equation (14):

p(x,y,t)=a(x,y,t)*v(x,y,t) (14)

The position of each blood vessel can be specified even when the data p (x, y, t) defined by the equation (14) instead of the equation (1) is used.

Many widely different embodiments of the invention may be configured without departing from the spirit and the scope of the present invention. It should be understood that the present invention is not limited to the specific embodiments described in the specification, except as defined in the appended claims.

1. A magnetic resonance imaging apparatus configured to execute a pulse sequence for generating a phase shift of each spin corresponding to a flow rate of the spin to thereby acquire magnetic resonance signals from a subject and to determine a position of each blood vessel of the subject based on each of the magnetic resonance signals, said magnetic resonance imaging apparatus comprising a blood vessel position specifying device configured to specify a position of each blood vessel based on a change in signal intensity of the magnetic resonance signal with time and based on a change in the flow rate of the spin with time.

2. The magnetic resonance imaging apparatus according to claim 1, wherein the blood vessel position specifying device is configured to specify the position of each blood vessel based on a change in the signal intensity of the magnetic resonance signal with time at each position of a predetermined cutting plane of the subject and the change in the flow rate of the spin with time at each position thereof.

3. The magnetic resonance imaging apparatus according to claim 2, wherein the blood vessel position specifying device is configured to specify the position of each blood vessel based on data depending on the signal intensity and the flow rate.

4. The magnetic resonance imaging apparatus according to claim 3, wherein a data sequence indicative of changes in the data with time is determined for each position of the predetermined cutting plane of the subject and the position of each blood vessel is specified based on the data sequence.

5. The magnetic resonance imaging apparatus according to claim 4, wherein the blood vessel position specifying device is configured to:
determine a maximum value in a time direction of an absolute value of the data in the data sequence for each position of the predetermined cutting plane; and
a specify the position of each blood vessel based on the maximum value.

6. The magnetic resonance imaging apparatus according to claim 4, wherein the blood vessel position specifying device is configured to specify the position of each blood vessel based on a correlation between the data sequences at positions adjacent to each other in the predetermined cutting plane.

7. The magnetic resonance imaging apparatus according to claim 5, wherein the blood vessel position specifying device is configured to specify the position of each blood vessel based on a correlation between the data sequences at positions adjacent to each other in the predetermined cutting plane.

8. The magnetic resonance imaging apparatus according to claim 4, wherein the blood vessel position specifying device is configured to:
determine means values or standard deviations of the data sequences at the positions adjacent to each other in the predetermined cutting plane; and
specify the position of the blood vessel based on the means values or the standard deviations.

9. The magnetic resonance imaging apparatus according to claim 5, wherein the blood vessel position specifying device is configured to:
determine means values or standard deviations of the data sequences at the positions adjacent to each other in the predetermined cutting plane; and
specify the position of the blood vessel based on the means values or the standard deviations.

10. The magnetic resonance imaging apparatus according to claim 6, wherein the blood vessel position specifying device is configured to:
determine means values or standard deviations of the data sequences at the positions adjacent to each other in the predetermined cutting plane; and
specify the position of the blood vessel based on the means values or the standard deviations.

11. The magnetic resonance imaging apparatus according to claim 4, wherein the blood vessel position specifying device is configured to:
determine a difference in time direction between the data for each data sequence; and
eliminate an artifact based on the difference.

12. The magnetic resonance imaging apparatus according to claim 5, wherein the blood vessel position specifying device is configured to:
determine a difference in time direction between the data for each data sequence; and
eliminate an artifact based on the difference.

13. The magnetic resonance imaging apparatus according to claim 6, wherein the blood vessel position specifying device is configured to:
determine a difference in time direction between the data for each data sequence; and
eliminate an artifact based on the difference.

14. The magnetic resonance imaging apparatus according to claim 7, wherein the blood vessel position specifying device is configured to:
determine a difference in time direction between the data for each data sequence; and
eliminate an artifact based on the difference.

15. The magnetic resonance imaging apparatus according to claim 1, wherein the blood vessel position specifying device is configured to eliminate noise based on the change in the signal intensity of the magnetic resonance signal with the time.

16. The magnetic resonance imaging apparatus according to claim 1, wherein the blood vessel position specifying device is configured to determine whether an extracted blood vessel is an artery or a vein based on the change in the flow rate with the time.

17. The magnetic resonance imaging apparatus according claim 2, further comprising an image generation device configured to generate an image having the data.

18. The magnetic resonance imaging apparatus according to claim 17, wherein the image generation device is configured to specify a position of a blood vessel based on at least two images of an intensity image indicative of the signal intensity of the magnetic resonance signal, a flow rate image indicative of the flow rate, and an image indicative of the data which depends on the signal intensity and the flow rate.

19. The magnetic resonance imaging apparatus according to claim 1, wherein the magnetic resonance signals are acquired using a phase contrast method.

20. A method for executing a pulse sequence for generating a phase shift of each spin corresponding to a flow rate of the spin, said method comprising:
acquiring magnetic resonance signals from a subject;
determining a position of each blood vessel of the subject based on each of the magnetic resonance signals; and
specifying a position of each blood vessel based on a change in signal intensity of the magnetic resonance signal with time and based on a change in the flow rate of the spin with time.
